(19) United States
(12) Patent Application Publication
(10) Pub. No.: US 2020/0408711 A1
(43) Pub. Date: Dec. 31, 2020

(54) METHOD OF MANUFACTURING SENSOR FOR DETECTING HYDROGEN PEROXIDE AND SENSOR FOR DETECTING HYDROGEN PEROXIDE MANUFACTURED BY THE SAME

(71) Applicant: KOREA INSTITUTE OF SCIENCE AND TECHNOLOGY, Seoul (KR)

(72) Inventors: Young Tae BYUN, Seoul (KR); Jae Sung LEE, Seoul (KR); Da Woon JEONG, Seoul (KR)

(73) Assignee: KOREA INSTITUTE OF SCIENCE AND TECHNOLOGY, Seoul (KR)

(21) Appl. No.: 16/703,379

(22) Filed: Dec. 4, 2019

(30) Foreign Application Priority Data
Jun. 25, 2019 (KR) 10-2019-0075361

(51) Int. Cl. G01N 27/414; B01J 21/18; G01N 33/00

(52) U.S. Cl. CPC G01N 27/4146; G01N 27/127; G01N 33/0036; B01J 21/185

(57) ABSTRACT

Disclosed are a method of manufacturing a sensor for detecting hydrogen peroxide, the method including preparing a substrate; forming a gas sensing part including carbon nanotubes and porphyrin nanofiber on the substrate; and forming an electrode on the substrate on which the gas detector has been formed, and a sensor for detecting hydrogen peroxide manufactured by the method. In accordance with the method of manufacturing a sensor for detecting hydrogen peroxide of the present disclosure, a step of forming a gas detector including carbon nanotubes and porphyrin nanofiber on a substrate is included, whereby a sensor capable of detecting hydrogen peroxide vapor at a sub-ppm level can be manufactured.

### CROSS-REFERENCE TO RELATED APPLICATION

This application claims priority to and the benefit of Korean Patent Application No. 2019-0075361, filed on Jun. 25, 2019, the disclosure of which is incorporated herein by reference in its entirety.

### BACKGROUND

### 1. Field of the Invention

The present disclosure relates to a method of manufacturing a sensor for detecting hydrogen peroxide and a sensor for detecting hydrogen peroxide manufactured by the same, and more particularly, a method of manufacturing a sensor capable of detecting hydrogen peroxide vapor at a sub-ppm level due to the inclusion of a step of forming a gas detector including carbon nanotubes and porphyrin nanofiber on a substrate, and a sensor for detecting hydrogen peroxide manufactured by the same.

### 2. Discussion of Related Art

Semiconductor sensors have attracted a lot of attention with their use for detecting harmful gases, explosive gases and toxic gases in various fields such as the living environment, industrial safety, health, defense and terrorism. In particular, hazardous chemical spills, which occur frequently at home and abroad, emphasize the need for compact semiconductor sensors with high sensitivity and high selectivity in industrial sites.

In such semiconductor sensors, various sensing materials such as metal oxide semiconductors, polymers, and carbon nanotubes can be used. Thereamong, since carbon nanotube-based sensors have advantages such as low cost, miniaturization, a simple process, and compatibility with electronic circuits, research thereinto is actively underway.

As a conventional technology related to such semiconductor sensors, Patent Document 1 (Korean Patent Application Publication No. 10-2011-0123559) discloses a gas sensor including a substrate; first and second electrodes disposed to be spaced from each other on the substrate; and a gas sensing part including a carbon nanotube powder applied between the first electrode and the second electrode to connect the first and second electrodes. According to Patent Document 1, the selectivity of a detection gas can be improved, and a carbon nanotube sensor having improved reaction and recovery rates can be provided. As examples of the detection gas, an oxidizing gas, a reducing gas, and volatile organic compounds (VOCs) were proposed.

However, the semiconductor sensor proposed in Patent Document 1 is disadvantageous in that hydrogen peroxide (H₂O₂) cannot be sensed. Since hydrogen peroxide (H₂O₂) is used in various fields such as food, drinking water, and pharmaceuticals, there is a need for a sensor capable of efficiently monitoring the same. In addition, as terror attacks using peroxide-based explosives have recently occurred in Europe, there is a need for research on a sensor capable of sensitively, effectively, and accurately detecting hydrogen peroxide so as to monitor terrorism early and thus prevent the same.

Meanwhile, Patent Document 2 (Korean Patent No. 10-1847507) as a conventional technology discloses a sensor for detecting hydrogen peroxide including a substrate; a gas detector that includes carbon nanotubes surface-modified with a carboxyl group and a porphyrin and is formed on the substrate; and an electrode formed on the gas detector, and a method of manufacturing the sensor. Patent Document 2 discloses that the inclusion of carbon nanotubes surface-modified with a carboxyl group and a porphyrin as gas sensing materials allows the provision of a semiconductor sensor having excellent sensitivity and selectivity to hydrogen peroxide vapor.

However, the sensor according to Patent Document 2 can detect hydrogen peroxide vapor at a concentration of 50 ppm or more, but is disadvantageous in that it is difficult to detect hydrogen peroxide vapor at a concentration of 50 ppm or less.

Therefore, there is a need for a method of manufacturing a semiconductor sensor capable of sensing hydrogen peroxide vapor at a low concentration, particularly hydrogen peroxide vapor even at a sub-ppm level.

### RELATED ART DOCUMENTS

### Patent Documents

(Patent Document 0001) KR 1020110123559 A

(Patent Document 0002) KR 101847507 B1

### SUMMARY OF THE INVENTION

Therefore, the present disclosure has been made in view of the above problems, and it is one object of the present disclosure to provide a method of manufacturing a sensor capable of detecting hydrogen peroxide vapor at a sub-ppm level due to the inclusion of a step of forming a gas detector including carbon nanotubes and porphyrin nanofiber on a substrate, and a sensor for detecting hydrogen peroxide manufactured by the same.

In accordance with an aspect of the present disclosure, the above and other objects can be accomplished by the provision of a method of manufacturing a sensor for detecting hydrogen peroxide, the method including preparing a substrate; forming a gas sensing part including carbon nanotubes and porphyrin nanofiber on the substrate; and forming an electrode on the substrate on which the gas detector has been formed.

In accordance with another aspect of the present disclosure, there is provided a sensor for detecting hydrogen peroxide manufactured according to the method.

### BRIEF DESCRIPTION OF THE DRAWINGS

The above and other objects, features and advantages of the present disclosure will become more apparent to those of ordinary skill in the art by describing exemplary embodiments thereof in detail with reference to the accompanying drawings, in which:

FIG. 1 is a schematic diagram illustrating a process of manufacturing a sensor using the nanocomposite of SWCNTs and porphyrin nanofibers for detecting hydrogen peroxide according to an embodiment of the present disclosure;

FIG. 2 is a schematic diagram illustrating a process of manufacturing a sensor using a heterojunction between SWCNTs and porphyrin nanofibers for detecting hydrogen peroxide according to an embodiment of the present disclosure;

FIG. 3 is a schematic diagram illustrating a process of manufacturing porphyrin nanofiber according to an embodiment of the present disclosure;

FIG. 4 illustrates a comparison of UV-vis spectra of a TiOTPyP solution (black line) prepared according to an example and an SDBS solution (red line) to which the TiOTPyP solution has been added dropwise;

FIG. 5 illustrates scanning electron microscope (SEM) of porphyrin nanofiber prepared according to an example;

FIG. 6 is a graph illustrating the concentration (ppm) of H₂O₂ vapor according to the concentration (wt %) of a H₂O₂ solution;

FIG. 7 is a schematic diagram illustrating an apparatus for analyzing the characteristics of a sensor manufactured according to an example;

FIG. 8 is a graph illustrating resistance values over time of a sensor manufactured according to an example for 0.1 ppm H₂O₂ vapor;

FIG. 9 is a graph illustrating resistance values over time of a sensor manufactured according to an example for 0.5 ppm H₂O₂ vapor;

FIG. 10 is a graph illustrating resistance values over time of a sensor manufactured according to an example for 1.0 ppm H₂O₂ vapor;

FIG. 11 is a graph illustrating resistance values over time of a sensor manufactured according to an example for 5.0 ppm H₂O₂ vapor;

FIG. 12 is a graph illustrating resistance values over time of a sensor manufactured according to an example for 10.0 ppm H₂O₂ vapor;

FIG. 13 is a graph illustrating a comparison of resistance values over time when each of 0.1 ppm H₂O₂ vapor and 0.2 ppm H₂O₂ vapor is applied to a sensor manufactured according to an example; and

FIG. 14 is a graph illustrating H₂O₂ vapor concentration-dependent responses of a sensor manufactured according to an example.

### DETAILED DESCRIPTION OF EXEMPLARY EMBODIMENTS

Now, the present disclosure will be described in more detail to help understand the present disclosure. Terms or words used in the specification and the following claims shall not be limited to common or dictionary meanings, and have meanings and concepts corresponding to technical aspects of the embodiments of the present disclosure so as to most suitably express the embodiments of the present disclosure. Accordingly, the configurations shown in the examples and drawings disclosed in the present specification are merely preferred embodiments of the present disclosure and do not represent the full technical spirit of the present disclosure. Therefore, it should be understood that various equivalents and modifications may have been present at a filing time of the present application.

An embodiment of the present disclosure provides a method of manufacturing a sensor for detecting hydrogen peroxide, the method including (1) preparing a substrate; (2) forming a gas detector including carbon nanotubes and porphyrin nanofiber on a substrate; and (3) forming an electrode on the substrate on which the gas detector has been formed.

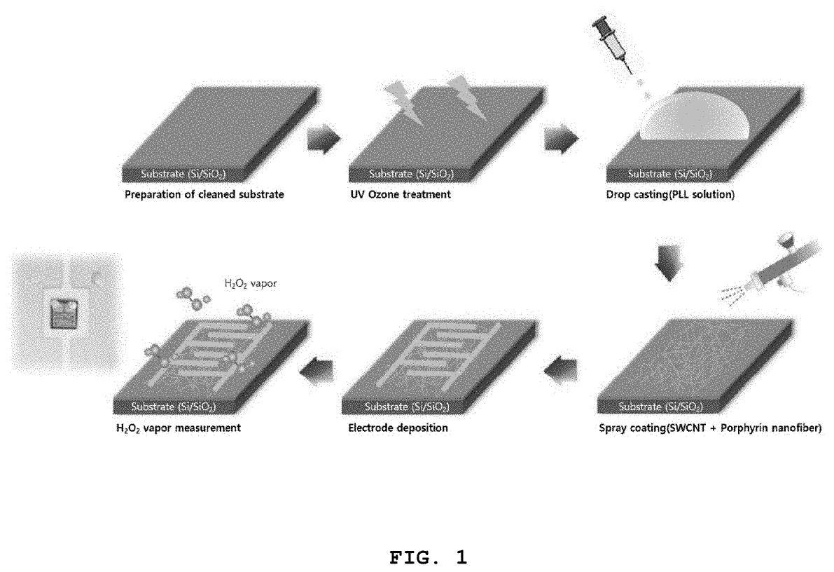

Hereinafter, with reference to FIGS. 1 to 3, each of the steps of a method of manufacturing a sensor for detecting hydrogen peroxide according to an embodiment of the present disclosure will be described in detail.

(1) Substrate Preparation Step

As materials of the substrate, III-V compound semiconductors such as Si, GaAs, InP, and InGaAs, glass, a thin oxide film, a dielectric thin film, a thin metal film, and the like may be used, but the present disclosure is not limited thereto. According to an embodiment of the present disclosure, the substrate may include a silicon substrate or a silicon substrate including an insulating film formed on a surface thereof. As a particular example, a silicon substrate (SiO₂/Si substrate) including a silicon oxide film (SiO₂) formed on a surface thereof may be included.

The insulating film may be formed on a substrate using a thermal oxidation method, a deposition method, a spin coating method, or the like, but the present disclosure is not limited thereto. In particular, in the case of a thermal oxidation method, a thermal insulating film may be formed by heating at 1000° C. or higher using a thermal diffusion furnace. In addition, in the case of a deposition method, a SiO₂ thin film may be formed using plasma-enhanced chemical vapor deposition (PECVD) or low-pressure chemical vapor deposition (LPCVD). When a spin coating method is used, a SiO₂ thin film may be formed using silica-on-glass (SOG) and the insulating film may be formed to a thickness of 120 to 300 nm.

According to an embodiment of the present disclosure, step (1) may include step (1-1) of hydrophilically modifying a surface of the substrate; and step (1-2) of coating a poly-L-lysine (PLL) solution on the substrate that has been subjected to step (1-1). After the steps, it is preferred to perform “step (2) of forming a gas detector” because a thin film, in which gas sensing materials, i.e., carbon nanotubes and porphyrin nanofiber, are uniformly distributed, may be formed on the substrate.

According to an embodiment of the present disclosure, in step (1-1), the modification may be performed by UV ozone treatment or oxygen plasma treatment. When a substrate surface is hydrophilized by the modification, wettability is improved, whereby a hydrophilic PLL solution may be easily coated on the substrate.

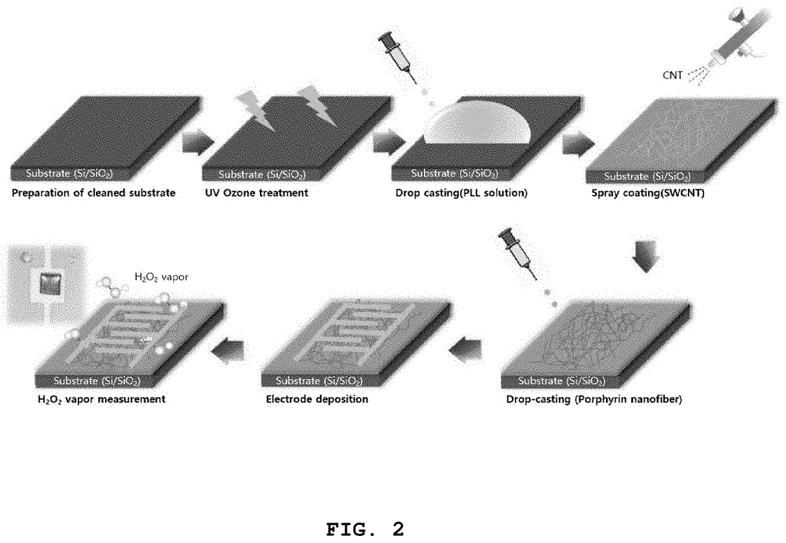

According to an embodiment of the present disclosure, coating with the PLL solution in step (1-2) may be performed by one or more methods selected from the group consisting of drop casting, spray coating, and spin coating. In particular, drop casting may be used. The drop casting process is a method of dropwise adding a PLL solution onto a substrate, followed by drying the same to evaporate a solvent. Here, as the solvent, water may be used. In particular, the third step (drop casting) of FIGS. 1 and 2 schematically illustrate a PLL solution added dropwise onto a substrate according to an embodiment of the present disclosure.

The main purpose of the PLL solution coating is to uniformly form a thin film, formed of carbon nanotubes and porphyrin nanofiber forming a gas detector, on a substrate. In particular, PLL is rich in active amino groups and has excellent cell adhesion and excellent solubility in water. When a gas detector including carbon nanotubes surface-modified with a carboxyl group is formed on a substrate surface uniformly coated with such a PLL solution, amino groups of the PLL are covalently bonded to carboxyl groups of the carbon nanotubes, thereby forming a gas detector having a solid and uniform thin film form.

(2) Gas Detector Forming Step

This step is a step of forming a gas detector including carbon nanotubes and porphyrin nanofiber on the substrate prepared according to step (1).

Carbon nanotubes (CNTs) are generally known to have 1000 times the current density of copper wires and 10 times the carrier mobility of silicon. Accordingly, carbon nanotubes are widely used as a material of high-response/high-speed electronic devices. In addition, a highly responsive chemical biosensor may be manufactured using a change in electrical conductivity due to an interaction between a sensing target material and carbon nanotubes. Since carbon nanotubes can operate at room temperature unlike existing metal oxide semiconductor sensors, and allow the miniaturization of a sensor size due to a nano-scale size thereof, they may be applied to a portable sensor.

According to an embodiment of the present disclosure, the carbon nanotubes may include one or more selected from the group consisting of single-walled carbon nanotubes (SWCNTs), double-walled carbon nanotubes, and multi-walled carbon nanotubes. In particular, the carbon nanotubes may include single-walled carbon nanotubes. When the single-walled carbon nanotubes are used as a sensing material, superior performance, compared to multi-walled carbon nanotubes, may be exhibited in terms of a response, a reaction speed, and the like.

According to an embodiment of the present disclosure, the carbon nanotubes may include carbon nanotubes surface-modified with a carboxyl group. When a gas detector including such carbon nanotubes surface-modified with a carboxyl group is formed on a substrate that has been subjected to a coating process with a PLL solution, carboxyl groups (—COOH) on surfaces of carbon nanotubes covalently bind to active amino groups of PLL, so that a gas detector having a solid and uniform thin film form may be formed on the substrate.

Meanwhile, since the present disclosure uses porphyrin nanofiber along with the carbon nanotubes as sensing materials, a semiconductor sensor exhibiting an excellent response when reacting with hydrogen peroxide vapor at a sub-ppm level may be provided. In particular, the porphyrin is a precursor compound of hemoglobin, chlorophyll, and a material related thereto and is a generic term for a macrocyclic compound wherein four pyrrole units are connected by methine groups. Such a porphyrin has a planar structure suitable for an π-stacking interaction with SWCN sidewalls. Accordingly, a porphyrin and a SWCNT are strongly and physically coupled to each other by van des Waals forces, so that a stable sensor may be manufactured.

According to an embodiment of the present disclosure, the porphyrin nanofiber may include oxo-[5,10,15,20-tetra(4-pyridyl)porphyrinato]titanium (IV) (TiOTPyP). Here, TiOTPyP may be represented by Formula 1 below:

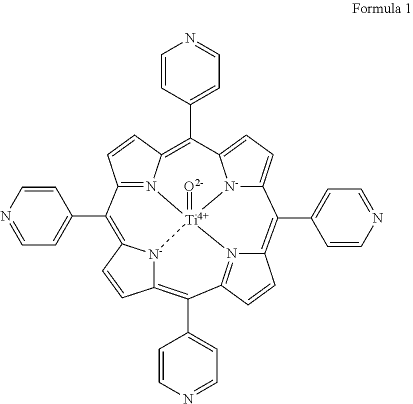

TiOTPyP is a kind of metalloporphyrin wherein TiO²+ is bound to a central void of a bivalent anion formed by the loss of two hydrogen ions inside a porphine. The TiO²⁺ couples with four ligands in an axial direction, thus forming a stable structure.

According to an embodiment of the present disclosure, the method of manufacturing the porphyrin nanofiber may include (a) a step of preparing a surfactant solution; (b) a step of dissolving a porphyrin in chloroform to prepare a porphyrin solution; (c) a step of dropwise adding the porphyrin solution to the surfactant solution, which is being stirred; (d) a step of evaporating chloroform from the mixture obtained according to step (c); and (e) a step of centrifuging the chloroform-evaporated mixture according to step (d). When the resultant nanofiber-type porphyrin is used as a sensing material, a response may be significantly improved, compared to cases of using irregular nanospecies or short nanorod-type porphyrin as a sensing material, whereby a sensor capable of detecting hydrogen peroxide vapor at a sub-ppm level may be provided.

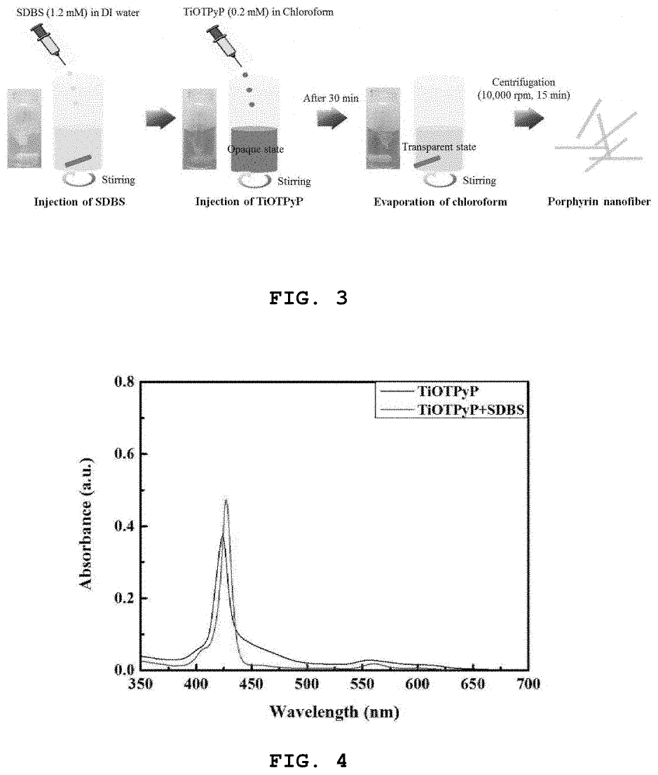

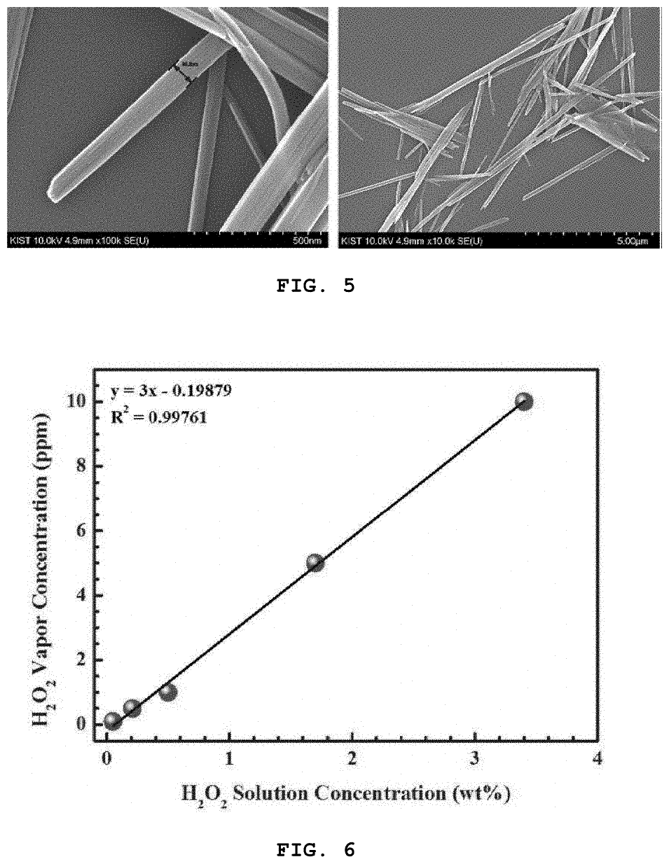

In particular, FIG. 3 schematically illustrates a process of manufacturing porphyrin nanofiber using the porphyrin represented by Formula 1 and, as the surfactant, sodium dodecylbenzenesulfonate (SDBS), according to an embodiment of the present disclosure. As shown in FIG. 3, when the porphyrin solution (TiOTPyP (0.2 mM) in chloroform) is added dropwise to the surfactant solution (SDBS (1.2 mM) in DI water), which is being stirred (step ┌Injection of TiOTPyP┘ in FIG. 3) according to step (c), an opaque solution may be obtained. For reference, FIG. 4 illustrates a comparison of UV-vis spectra of a TiOTPyP solution (black line) and an SDBS solution (red line) to which the TiOTPyP solution has been added dropwise. Next, when chloroform is evaporated from the opaque solution obtained according to step (c), a nanofiber-type porphyrin may be formed in the solution as the solution gradually changes to a transparent state. Finally, when the transparent solution is centrifuged, porphyrin nanofiber may be obtained. Next, a washing process of adding Milli-Q Water to the porphyrin nanofiber and centrifuging the same several times may be further performed. SEM images of the porphyrin nanofiber manufactured according to the embodiment of the present disclosure are illustrated in FIG. 5. A left photograph of FIG. 5 is a 10× magnification of a right photograph thereof.

A method of forming a gas detector according to the present disclosure may be selected from a first method of forming the gas detector in a single layer and a second method of forming the gas detector in two layers.

First, FIG. 1 schematically illustrates a process of manufacturing a sensor for detecting hydrogen peroxide according to the first method. According to the embodiment of the present disclosure shown in FIG. 1, step (2) may include a step of coating a dispersing solution including carbon nanotubes and porphyrin nanofiber on the substrate. Here, a carbon nanotube-porphyrin nanofiber thin film may be formed on the substrate. According to this method, carbon nanotubes are mixed with porphyrin nanofiber, and then coated to form a thin film for a hydrogen peroxide sensor. Accordingly, the method may contribute to the establishment of process conditions suitable for mass production.

According to an embodiment of the present disclosure, the dispersing solution including carbon nanotubes and porphyrin nanofiber may include one or more dispersion media selected from the group consisting of deionized water (DI water) and Milli-Q Water. In addition, carbon nanotubes and porphyrin nanofiber in the dispersing solution may be uniformly dispersed by irradiating the dispersing solution with ultrasonic waves. Here, the concentration of carbon nanotubes in the dispersing solution may be 0.01 to 0.50 mg/ml, and the concentration of porphyrin nanofiber therein may be 0.5 to 1.5 mg/ml. When the concentrations of the carbon nanotubes and the porphyrin nanofiber are respectively within the ranges, a sensor having excellent selectivity to hydrogen peroxide vapor and being capable of detecting hydrogen peroxide vapor even at a sub-ppm level may be manufactured.

According to an embodiment of the present disclosure, the dispersing solution including carbon nanotubes and porphyrin nanofiber may be coated by one or more methods selected from the group consisting of drop casting, spray coating and spin coating, particularly spray coating was used. For example, FIG. 1 schematically illustrates a manufacturing process of applying the dispersing solution by spray coating according to an embodiment of the present disclosure.

Next, FIG. 2 schematically illustrates a process of manufacturing a sensor for detecting hydrogen peroxide according to the second method. According to the embodiment of the present disclosure shown in FIG. 2, step (2) may include step (2-1) of adsorbing the carbon nanotubes on the substrate to form a first sensing layer; and step (2-2) of coating porphyrin nanofiber on the first sensing layer to form a second sensing layer.

According to an embodiment of the present disclosure, the adsorption of the carbon nanotubes in step (2-1) may be performed by one or more methods selected from the group consisting of a dipping method of dipping a substrate in a solution, in which the carbon nanotubes are dispersed, and then taking the substrate out of the solution and a spray method of spraying a solution, in which the carbon nanotubes are dispersed, onto a substrate. Of the methods, when the spray method is used, it is possible to more uniformly disperse carbon nanotubes, and a process suitable for mass production may be provided. Such a spray process may be performed under an argon (Ar) atmosphere to prevent oxidation between oxygen and carbon nanotubes.

According to an embodiment of the present disclosure, the solution in which carbon nanotubes are dispersed may include one or more dispersion media selected from the group consisting of dichlorobenzene, ortho-dichlorobenzene, N-methyl-2-pyrrolidinone, hexamethylphosphoramide, monochlorobenzene, N,N-dimethylformamide, dichloroethane, isopropyl alcohol, ethanol, chloroform, and toluene. In addition, the carbon nanotubes may be uniformly dispersed by irradiating the solution, in which the carbon nanotubes are dispersed, with ultrasonic waves. The concentration of the carbon nanotubes in the solution in which the carbon nanotubes are dispersed may be 0.01 to 0.50 mg/ml. When the concentration of the carbon nanotubes is within the range, it is preferable for manufacturing an economic sensor having excellent selectivity.

According to an embodiment of the present disclosure, in step (2-2), the porphyrin nanofiber may be coated on the first sensing layer in an aqueous dispersing solution state. Here, the second sensing layer may be formed in a thin film form having a thickness of 5 to 100 nm on the first sensing layer. In the aqueous dispersing solution, the concentration of porphyrin nanofiber may be 0.02 to 0.1 mg/ml. When the concentration of the porphyrin nanofiber is within the range, it is preferable for manufacturing an economic sensor having an excellent detection effect for hydrogen peroxide vapor at a sub-ppm level. In addition, the aqueous dispersing solution of the porphyrin nanofiber may include one or more dispersion media selected from the group consisting of deionized water (DI water) and Milli-Q Water.

According to an embodiment of the present disclosure, in step (2-2), the porphyrin nanofiber may be coated by one or more methods selected from the group consisting of drop casting, spray coating, and spin coating, particularly drop casting was used.

As the method of forming a gas detector according to the present disclosure, the first method or the second method may be used, but the first method may be economically more efficient because it is simpler than the second method.

(3) Electrode Formation Step

This step is a step of forming an electrode on the substrate on which the gas detector has been formed according to step (2). Here, the electrode may be a source electrode and a drain electrode. As a material of the electrode, at least one metal of gold (Au), silver (Ag), chromium (Cr), tantalum (Ta), titanium (Ti), copper (Cu), aluminum (Al), molybdenum (Mo), tungsten (W), nickel (Ni), palladium (Pd), and platinum (Pt) may be used.

The electrode may be formed according to a general photolithography process or a process using a shadow mask. When a photolithography process is used, a photoresist is formed on the substrate that has been subjected to steps (1) and (2), and then a region at which a source electrode and a drain electrode are to be formed is exposed through an exposure process, and then an electrode is deposited using a general metal and metal oxide deposition apparatus such as a thermal evaporator, an e-beam evaporator, or a sputter, and then the photoresist is removed with a photoresist stripper, thereby forming source and drain electrodes formed of a metal and a metal oxide. When a shadow mask is used, the shadow mask is brought into contact with the substrate that has been subjected to steps (1) and (2), and then source and drain electrodes may be formed through the aforementioned electrode deposition process.

Meanwhile, another embodiment of the present disclosure provides a sensor for detecting hydrogen peroxide manufactured according to the method. In particular, the sensor for detecting hydrogen peroxide may include a substrate; a gas detector that is formed on the substrate and includes carbon nanotubes and porphyrin nanofiber; and an electrode formed on the gas detector. Characteristics of each of the components included in the substrate, the gas detector, and the electrode are the same as those described above.

When the gas detector including carbon nanotubes and porphyrin nanofiber is used, a semiconductor sensor exhibiting an excellent response when reacting with hydrogen peroxide vapor even at a sub-ppm level may be provided. Therefore, the sensor for detecting hydrogen peroxide according to the present disclosure may be applied in various fields such as biochemistry, food chemistry, photochemistry, pharmaceuticals, biomedicine, health, and terror prevention.

Hereinafter, the present disclosure will be described in more detail with reference to Examples, but the present disclosure is not limited to these Examples.

### Example

1. Porphyrin Nanofiber Preparation (See FIG. 3)

First, sodium dodecylbenzenesulfonate (SDBS, manufacturer: Tokyo Chemical Industry Co., Ltd (TCI), trade name: Dodecene-1 LAS, purity: >98.0%, CAS No: 25155-30-0) was dissolved in deionized water (DI water) to prepare a 1.2 mM SDBS solution.

Next, a porphyrin (oxo-[5,10,15,20-tetra(4-pyridyl)porphyrinato]titanium (IV), TiOTPyP, Tokyo Chemical Industry Co., LTD., CAS No. 105250-49-5) was dissolved in chloroform (Sigma-Aldrich, Anhydrous, >99%) as a solvent, thereby preparing a 0.2 mM TiOTPyP solution.

Next, 10 mL of the SDBS solution was added dropwise to a 20 mL glass vial while stirring. After terminating the addition of the SDBS solution, 800 μL of the TiOTPyP solution was added dropwise to the 10 mL SDBS solution, which was being stirred. In this case, an opaque solution was obtained in the container due to the dropping of the TiOTPyP solution.

Next, the opaque solution obtained after completing the dropping of the porphyrin solution was stirred for 30 minutes to evaporate chloroform in the opaque solution. In this case, the opaque solution was changed into a transparent solution as the chloroform was evaporated.

Next, the transparent solution was centrifuged at 10,000 rpm for 15 minutes, thereby obtaining porphyrin nanofiber. SEM photographs of the obtained porphyrin nanofiber are illustrated in FIG. 5.

2. Manufacture of Sensor for Detecting Hydrogen Peroxide (See FIG. 1)

A silicon oxide insulating film (SiO₂) was formed on a silicon substrate, thereby preparing a SiO₂/Si substrate. Here, the thickness of the insulating film was 300 nm.

Next, the substrate was subjected to UV ozone treatment for 20 minutes to hydrophilically modify a surface of the substrate, and then the surface of the substrate was treated with a poly-L-lysine (PLL) solution (Sigma Aldrich, 0.1% (w/v) in H₂O) for 20 minutes in a drop casting manner and, after 20 minutes, was dried by blowing with a nitrogen gun.

Next, 5 mg of porphyrin nanofiber manufactured according to the process ┌1. Porphyrin nanofiber preparation┘ was fed into 5 ml of a single-walled carbon nanotube (SWCNT) solution (Nano Integris, IsoNanotubes-S 95%) having a concentration of 0.01 mg/ml, followed by sonication at room temperature for 4 hours, thereby preparing a dispersing solution in which porphyrin nanofiber and SWCNT were uniformly dispersed. Here, deionized water (DI water) was used as a solvent of the SWCNT solution, and the SWCNTs were single-walled carbon nanotubes surface-modified with a carboxyl group.

Next, 4 ml of the dispersing solution was sprayed on the substrate surface treated with the PLL solution using an air-brush-spray gun (manufacturer: Mr. Hobby, model name: PS-770) equipped with a 0.18 mm nozzle so that porphyrin nanofiber and SWCNTs were adsorbed into the substrate. Here, the dispersing solution was sprayed under an argon (Ar) atmosphere. Next, to remove the dispersing solution including porphyrin nanofiber and SWCNTs not adsorbed onto the substrate, the substrate was washed with distilled water and dried using nitrogen gas.

Next, a source electrode and a drain electrode were formed on the dried substrate according a general photolithography process. Here, as the source and drain electrodes, Au (200 nm) was used.

Evaluation Example: Sensor Characteristic Analysis

In the case of an aqueous H₂O₂ solution, a liquid state and a vapor state coexist at room temperature under atmospheric pressure (25° C., 1 atm). Accordingly, first, to investigate the concentration (ppm) of H₂O₂ vapor according to the concentration (wt %) of an aqueous H₂O₂ solution, an aqueous H₂O₂ solution at each of concentrations of 0.05 wt %, 0.21 wt %, 0.5 wt %, 1.7 wt %, and 3.4 wt % was prepared. Next, 144 ml of the aqueous H₂O₂ solution at each concentration was contained in a 500 ml flask vessel at room temperature under atmospheric pressure (25° C., 1 atm), and the concentration of H₂O₂ vapor in a vapor phase in the flask vessel was analyzed using a gas concentration analyzer (ANALYTICAL TECHNOLOGY, PORTASENS II). In addition, analysis results of the concentration of H₂O₂ vapor according to the concentration of the aqueous H₂O₂ solution are shown in Table 1 and FIG. 6.

TABLE 1

| Concentration (wt %) | Concentration (ppm) |
|---|---|
| of aqueous H₂O₂ | of H₂O₂ vapor |
| 0.05 | 0.1 |
| 0.21 | 0.5 |
| 0.5 | 1.0 |
| 1.7 | 5.0 |
| 3.4 | 10.0 |

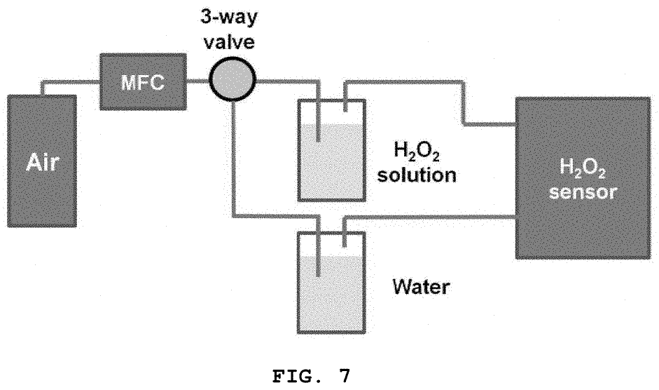

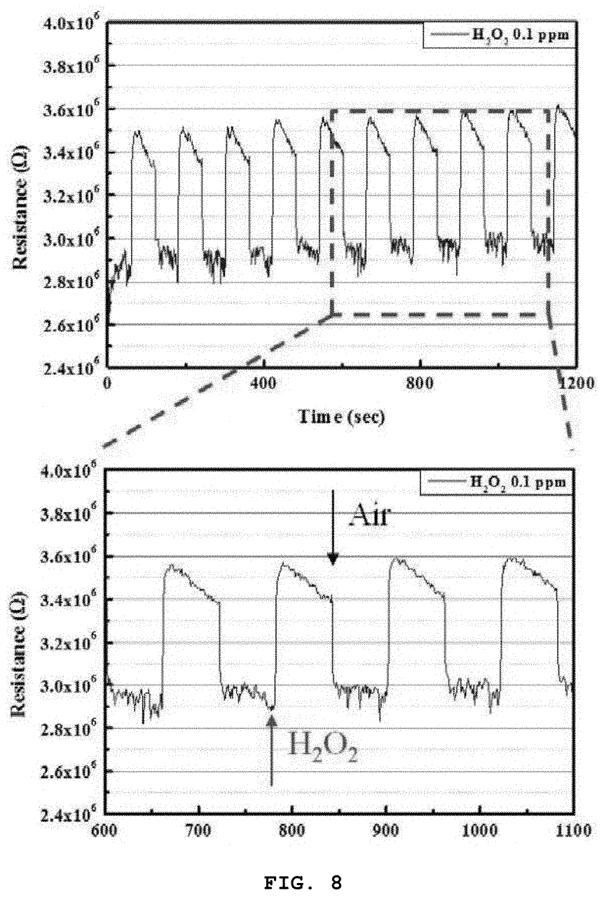

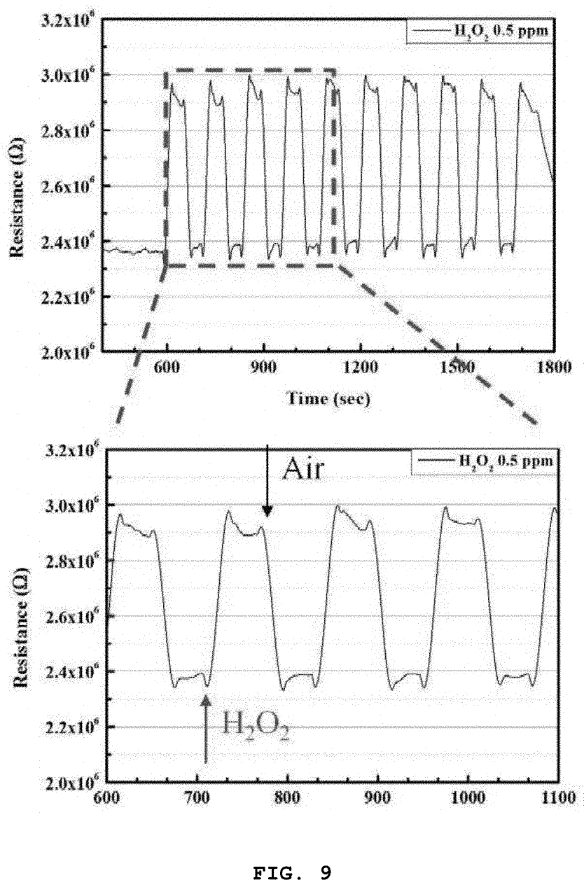

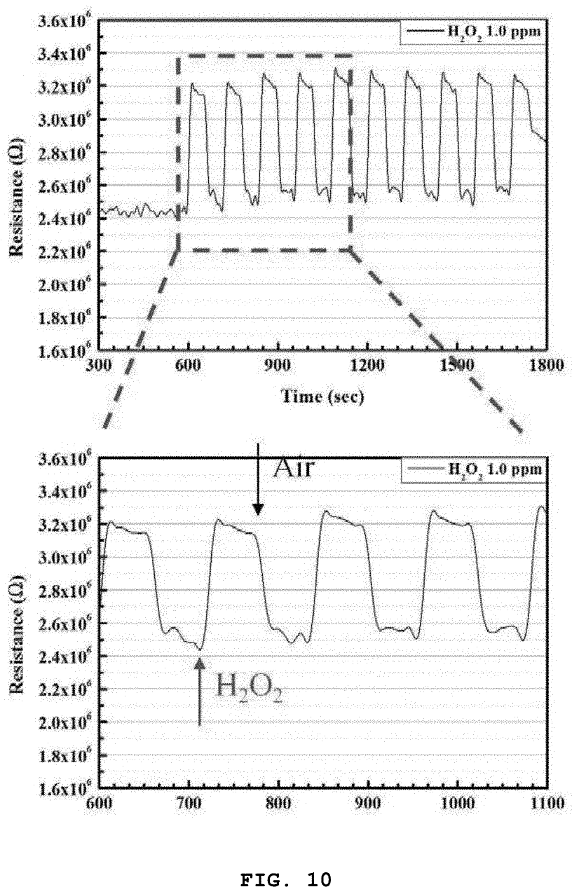

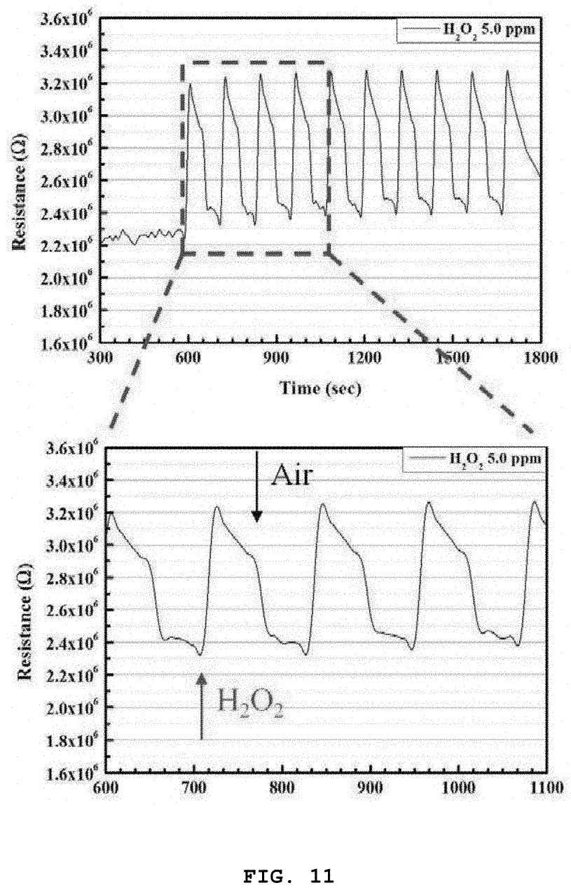

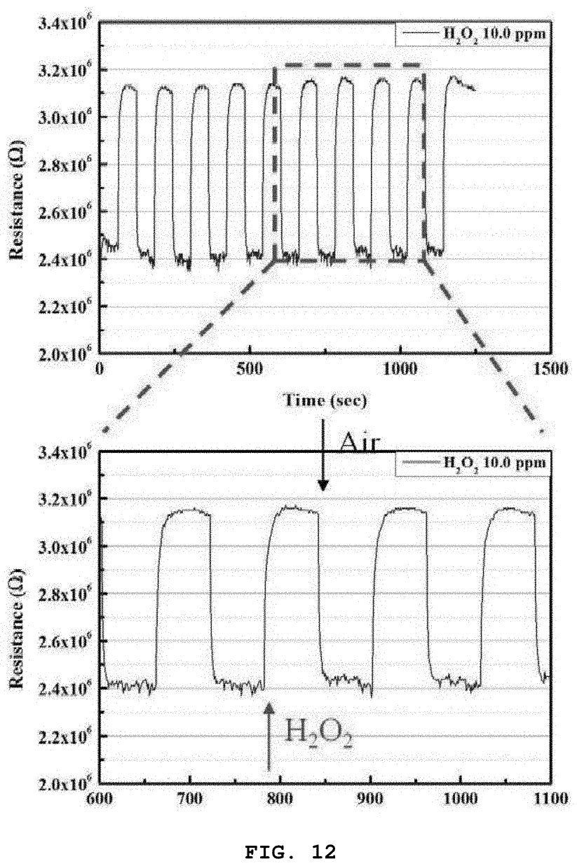

Next, the characteristics of the sensor manufactured according to the example were analyzed using the apparatus illustrated in FIG. 7. In particular, the sensor was connected to a DC power supply (Keithley 2400), and then hydrogen peroxide (H₂O₂) vapor was flowed using a mass flow controller (MFC). A resistance change in a sensing body was measured while applying constant DC power. Measurement results are illustrated in FIGS. 8 to 13. Here, measurement conditions were as follows;
Total flow: 300 sccm
Bias voltage: 1.0 V
Balance gas: H₂O vapor in Air
Gas injection time: 1 minute
Recovery time: 1 minute

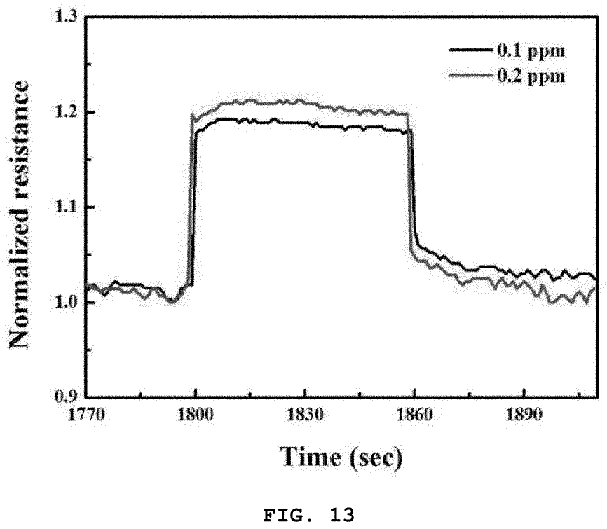

FIGS. 8 to 12 respectively illustrate resistance changes measured while supplying H₂O₂ vapor at each of concentrations of 0.1 ppm, 0.5 ppm, 1.0 ppm, 5.0 ppm and 10.0 ppm to a sensor. FIG. 13 is a graph illustrating a comparison of resistance values over time when H₂O₂ vapor at each of concentrations of 0.1 ppm and 0.2 ppm was applied. Examining FIGS. 8 to 13, it can be confirmed that the sensor manufactured according to the present disclosure is a highly responsive sensor capable of detecting H₂O₂ vapor at 10.0 ppm or less and H₂O₂ vapor even at a sub-ppm level.

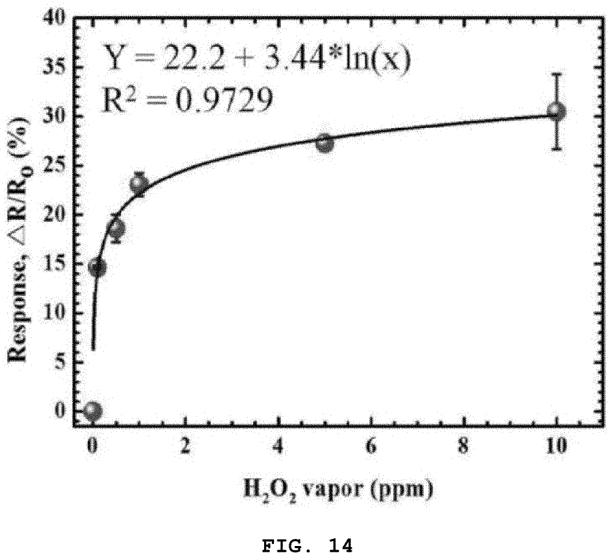

In addition, a response (gas response) of the sensor manufactured according to the example was calculated according to the following Equation (1). Results are shown in Table 2 and FIG. 14.

Response (%)=(ΔR/R₀)×100 (1)

wherein R₀ denotes an initial resistance value when there is no reaction gas, and ΔR denotes a value obtained by subtracting R₀ from a resistance value when there is a reaction gas.

TABLE 2

| Concentration (ppm) | Response |
|---|---|
| of H₂O₂ vapor | (%) |
| 0.1 | 14.65 |
| 0.2 | 16.5 |

Examining Table 2, it can be confirmed that the sensor manufactured according to the examples of the present disclosure may detect H₂O₂ vapor even at sub-ppm levels such as 0.1 ppm and 0.2 ppm. In addition, examining FIG. 14 illustrating a graph representing a H₂O₂ vapor concentration-dependent response change, it can be confirmed that a response of the sensor increases with an increasing concentration, and, when repeatedly measured, an error margin of each concentration is very small.

In accordance with a method of manufacturing a sensor for detecting hydrogen peroxide of the present disclosure, a step of forming a gas detector including carbon nanotubes and porphyrin nanofiber on a substrate is included, whereby a sensor capable of detecting hydrogen peroxide vapor at a sub-ppm level can be manufactured.

In addition, since a sensor for detecting hydrogen peroxide manufactured according to the present disclosure can be operated operate at room temperature without a heater, it is possible to minimize power consumption. Further, since the sensor can be miniaturized, it can be used as a portable device.

Although the present disclosure has been described through non-limiting examples and figures, the technical spirit of the present disclosure is not intended to be limited to the examples and drawings. The scope of the disclosure is defined not by the detailed description of the disclosure but by the appended claims, and all technical ideas within the equivalent scope will be construed as within the scope of the present disclosure.

What is claimed is:

1. A method of manufacturing a sensor for detecting hydrogen peroxide, the method comprising:
preparing a substrate;
forming a gas sensing part comprising carbon nanotubes and porphyrin nanofiber on the substrate; and
forming an electrode on the substrate on which the gas detector has been formed.

2. The method according to claim 1, wherein the substrate comprises a silicon substrate.

3. The method according to claim 1, wherein the substrate comprises a silicon substrate having a surface on which a silicon oxide film is formed.

4. The method according to claim 1, wherein the preparing comprises:
hydrophilically modifying a surface of the substrate; and
coating the hydrophilically modified substrate with a poly-L-lysine (PLL) solution.

5. The method according to claim 4, wherein the hydrophilically modifying is performed by UV ozone treatment or oxygen plasma treatment.

6. The method according to claim 4, wherein the coating is performed by one or more methods selected from the group consisting of drop casting, spray coating, and spin coating.

7. The method according to claim 1, wherein the carbon nanotubes comprise single-walled carbon nanotubes (SWCNTs).

8. The method according to claim 1, wherein the carbon nanotubes comprise carbon nanotubes surface-modified with a carboxyl group.

9. The method according to claim 1, wherein the porphyrin nanofiber comprises oxo-[5,10,15,20-tetra(4-pyridyl)porphyrinato]titanium(IV).

10. The method according to claim 1, wherein a method of manufacturing the porphyrin nanofiber comprises:
preparing a surfactant solution;
dissolving porphyrin in chloroform to prepare a porphyrin solution;
dropwise adding the porphyrin solution to the surfactant solution being stirred;
evaporating chloroform from a mixture obtained according to the adding; and
centrifuging the mixture from which chloroform has been evaporated.

11. The method according to claim 1, wherein the forming of the gas sensing part comprises coating the substrate with a dispersing solution comprising carbon nanotubes and porphyrin nanofiber.

12. The method according to claim 11, wherein the dispersing solution comprising carbon nanotubes and porphyrin nanofiber comprises one or more dispersion media selected from the group consisting of deionized water (DI water) and Milli-Q Water.

13. The method according to claim 11, wherein the dispersing solution comprising carbon nanotubes and porphyrin nanofiber is applied by one or more methods selected from the group consisting of drop casting, spray coating and spin coating.

14. The method according to claim 1, wherein the forming of the gas sensing part comprises:
adsorbing the carbon nanotubes onto the substrate to form a first sensing layer; and
coating the first sensing layer with porphyrin nanofiber to form a second sensing layer.

15. The method according to claim 14, wherein the adsorbing is performed by one or more methods selected from the group consisting of a dipping method of dipping a substrate in a solution, in which the carbon nanotubes are dispersed, and then taking the substrate out of the solution; and a spray method of spraying a solution, in which the carbon nanotubes are dispersed, onto a substrate.

16. The method according to claim 15, wherein the solution, in which the carbon nanotubes are dispersed, comprises one or more dispersion media selected from the group consisting of dichlorobenzene, ortho-dichlorobenzene, N-methyl-2-pyrrolidinone, hexamethylphosphoramide, monochlorobenzene, N,N-dimethylformamide, dichloroethane, isopropyl alcohol, ethanol, chloroform, and toluene.

17. The method according to claim 14, wherein, in the coating, the first sensing layer is coated with the porphyrin nanofiber in an aqueous dispersing solution state.

18. The method according to claim 14, wherein the coating is performed by one or more methods selected from the group consisting of drop casting, spray coating, and spin coating.

19. A sensor for detecting hydrogen peroxide manufactured according to the method of claim 1, comprising:
a substrate;
a gas detector formed on the substrate and configured to comprise carbon nanotubes and porphyrin nanofiber; and
an electrode formed on the gas detector.
